(19) United States
(12) Patent Application Publication
(10) Pub. No.: US 2023/0340703 A1
(43) Pub. Date: Oct. 26, 2023

(54) METHOD FOR PREPARING PALLADIUM-LOADED HETEROJUNCTION COMPOSITE FRAMEWORK AEROGEL AND METHOD FOR PREPARING HYDROGEN SENSOR

(71) Applicant: YANGTZE DELTA REGION INSTITUTE (QUZHOU), UNIVERSITY OF ELECTRONIC SCIENCE AND TECHNOLOGY OF CHINA, Quzhou (CN)

(72) Inventors: Xiongbang WEI, Quzhou (CN); Wei CHEN, Quzhou (CN); Jiaxuan LIAO, Quzhou (CN); Luxi SUI, Quzhou (CN); Yuhong QIU, Quzhou (CN); Xin LONG, Quzhou (CN)

(21) Appl. No.: 18/343,251

(22) Filed: Jun. 28, 2023

(30) Foreign Application Priority Data
Feb. 8, 2023 (CN) 202310083369.2

(51) Int. Cl. D01F 8/16; D01F 8/18; D01D 1/02; D01D 5/00; G01N 33/00; G01N 27/12

(52) U.S. Cl. CPC D01F 8/16; D01F 8/18; D01D 1/02; D01D 5/003; G01N 33/005; G01N 27/127; D10B 2331/14; D10B 2401/10

(57) ABSTRACT

A method for preparing a palladium-loaded heterojunction composite framework aerogel, including: preparing a hollow tin dioxide (SnO₂) nanofiber; preparing a tetrabutyl titanate-hollow SnO₂ nanofiber mixed solution; preparing a palladium dichloride (PdCl₂) precursor solution; adding the PdCl₂ precursor solution to the tetrabutyl titanate-hollow SnO₂ nanofiber mixed solution to form a heterojunction double-network composite framework gel; and preparing a palladium nanoparticle-loaded heterojunction double-network composite framework aerogel. A method for preparing a hydrogel sensor coated with the palladium-loaded heterojunction composite framework aerogel is also provided herein.

### CROSS-REFERENCE TO RELATED APPLICATIONS

This application claims the benefit of priority from Chinese Patent Application No. 202310083369.2, filed on Feb. 8, 2023. The content of the aforementioned application, including any intervening amendments thereto, is incorporated herein by reference in its entirety.

### TECHNICAL FIELD

This application relates to molecular sensors, and more particularly to a method for preparing a palladium-loaded heterojunction composite framework aerogel, and a method for preparing a hydrogen sensor.

### BACKGROUND

The development and utilization of green energy has received considerable attention in recent years. As a low-carbon and zero-carbon energy, hydrogen energy has achieved rapid development. Hydrogen provides the energy support for global sustainable development, and is employed as an important industrial chemical and green energy in various fields, such as automobiles, fuel cells, rocket engines, and chemical industry. Additionally, hydrogen also plays an effective role in the treatment of diseases, and exhibits a promising application prospect in the fields of medicine and biology. However, hydrogen is a colorless and odorless gas with a high energy density (120-140 MJ/kg), and is highly flammable and explosive when reaching the limiting concentration (4% in air). In this case, extremely strict safety standards are formulated for the storage, transportation and use of hydrogen. In order to further promote the application of hydrogen energy, it is necessary to eliminate the potential safety hazards existing in the production, storage and transportation of hydrogen. In other words, the design and development of hydrogen sensors is the basis for the development of hydrogen energy. Therefore, it is urgently required to develop a hydrogen sensor with high sensitivity, fast response-recovery characteristic, and strong stability.

In the existing technology, some hydrogen-sensitive aerogel materials have a single structure, and fails to meet the higher hydrogen-sensitive requirements. Some composite materials are prepared by simple physical compounding (e.g., vapor deposition and magnetron sputtering) of two ingredients, and these materials are superior to those structurally-single materials in properties. Unfortunately, the physical methods fail to optimize the nanoparticle size, and form mesoporous structures, so that the sieving effect for gas molecules is poor, and the specific surface area is far lower than the aerogel structure. Therefore, it fails to form more active sites at the surface and interior of the oxide, which is not conducive to the adsorption and desorption of the gas to be measured and oxygen.

### SUMMARY

An objective of this application is to provide a method for preparing a palladium-loaded heterojunction composite framework aerogel, and a method for preparing a hydrogen sensor.

In a first aspect, this application provides a method for preparing a palladium-loaded heterojunction composite framework aerogel, comprising:
(1) preparing a hollow tin dioxide (SnO₂) nanofiber;
(2) grinding the hollow SnO₂ nanofiber followed by addition of a tetrabutyl titanate-ab solute ethanol mixture and stirring at room temperature to form a tetrabutyl titanate-hollow SnO₂ nanofiber mixed solution; wherein a volume ratio of tetrabutyl titanate to absolute ethanol in the tetrabutyl titanate-absolute ethanol mixture is 1:(20-25); and a mass ratio of the hollow SnO₂ nanofiber to the tetrabutyl titanate-absolute ethanol mixture is 1:(50-100);
(3) preparing a palladium dichloride (PdCl₂) precursor solution with a pH ranging from 1.9 to 4.7;
(4) dropwise adding the PdCl₂ precursor solution prepared in step (3) to the tetrabutyl titanate-hollow SnO₂ nanofiber mixed solution prepared in step (2) at a rate of 1-2 drops/s under stirring at room temperature until a crude gel is formed; and subjecting the crude gel to aging and multiple solvent replacements with anhydrous ethanol to form a heterojunction double-network composite framework gel; and
(5) placing the heterojunction double-network composite framework gel inside a supercritical drying kettle, followed by immersion in absolute ethanol, and adjusting a temperature and pressure in the supercritical drying kettle to allow carbon dioxide gas inside the supercritical drying kettle to reach a supercritical fluid state;
regulating the pressure in the supercritical drying kettle such that a three-dimensional (3D) network structure of the heterojunction double-network composite framework gel is maintained in carbon dioxide gas; subjecting the heterojunction double-network composite framework gel to degassing and pressure holding to allow palladium ions to grow into palladium nanoparticles in situ, so as to form a palladium nanoparticle-loaded heterojunction double-network composite framework aerogel.

In an embodiment, in step (1), the palladium-loaded heterojunction composite framework aerogel is prepared through the following steps:
dissolving tin dichloride monohydrate (SnCl₂·H₂O) in a formamide-ethanol-acetone mixture, followed by stirring to form a SnO₂ precursor solution; wherein a volume ratio of formamide to ethanol to acetone in the formamide-ethanol-acetone mixture is 2.5:2.5:1; and a weight-volume ratio of the SnCl₂·H₂O to the formamide-ethanol-acetone mixture is 1 (g) 32-38 (mL);
dissolving polyvinylpyrrolidone (PVP) powder in the SnO₂ precursor solution followed by heating at 45-55° C. under stirring for at least 4 h for complete dissolution of the PVP powder to form a PVP-SnO₂ spinning solution; wherein a weight ratio of SnCl₂·H₂O to the PVP powder is (2-3):1;
subjecting the PVP-SnO₂ spinning solution to electrospinning to obtain a PVP-SnO₂ nanofiber mat, wherein the PVP-SnO₂ nanofiber mat has a fiber diameter of 20˜100 nm, and a specific surface area of 13˜17 m²/g; and
subjecting the PVP-SnO₂ nanofiber mat to calcination in a temperature-programmed furnace at 480-515° C. to obtain the hollow SnO₂ nanofiber, wherein the hollow SnO₂ nanofiber has a single tetragonal structure.

In an embodiment, in step (3), the PdCl₂ precursor solution is prepared through steps of:
dissolving PdCl₂ powder in concentrated hydrochloric acid followed by standing to obtain a chloropalladium acid solution;
adding a formamide-ethanol-deionized water mixture into the chloropalladium acid solution, followed by stirring at room temperature for 2 h to obtain an orange-brown transparent solution; wherein a weight ratio of the PdCl₂ powder to the concentrated hydrochloric acid is 1:(1-5); and a volume ratio of formamide to ethanol to deionized water in the formamide-ethanol-deionized water mixture is 1:(13˜16):(2˜2.5); and
adding PVP powder into the orange-brown transparent solution followed by stirring at room temperature and ultrasonic dispersion to obtain the PdCl₂ precursor solution.

In a second aspect, this application provides a method for preparing a hydrogen sensor, comprising:
preparing a gold interdigital electrode;
preparing a palladium-loaded heterojunction composite framework aerogel according to the method mentioned above; grinding the palladium-loaded heterojunction composite framework aerogel to obtain a nano powder; mixing the nano powder with deionized water to obtain a coating, wherein a weight ratio of the nano powder to the deionized water is 1:(10˜20); and

In an embodiment, the gold interdigital electrode is prepared by ion sputtering; wherein the ion sputtering is performed through steps of:
sputtering a gold target at a working distance of 25 mm and an electric current of 10 mA to generate gold ions; and allowing the gold ions to pass through a shadow mask to reach an aluminum oxide substrate and form a gold film on the aluminum oxide substrate, so as to form the gold interdigital electrode.

In this application, two or more metal-oxide-semiconductor-field-effect tube materials (MOS materials) are combined to form a ‘point-line’ heterojunction on a microscale by an electrostatic spinning technology and a sol-gel method, so as to realize the complementary advantages, and improve the sensing performance. Due to the enhanced catalytic activity, the formation of electron depletion layer, more adsorption sites and the change of band structure caused by heterojunction improves the response of hydrogen sensor, thereby improving and the sensitivity and response speed of gas sensing materials.

Compared with the prior art, this application has the following beneficial effects.

The present application combines the three-dimensional network structural characteristics of TiO₂ aerogel with the structural characteristics of hollow SnO₂ nanofibers. Based on electrospinning technology and sol-gel method, a “point-line” semiconductor heterogeneous structure is constructed, which has higher electron mobility compared with a single oxide semiconductor structure. In this case, it is suitable for the effective transport of carriers after adsorption of the to-be-measured gas. Moreover, the resistance signal of the “point-line” semiconductor heterogeneous structure changes significantly.

The three-dimensional network structure of the TiO₂ aerogel is employed as the primary network structure, and the added hollow SnO₂ nanofibers is used as the secondary network structure, which enhances the overall structural strength of the composite aerogel. Due to the hollow-tube structural characteristics of the hollow SnO2 nanofibers, the contact area between the composite aerogel and the to-be-measured gas is enlarged, and the transmission channel of the gas molecules to be detected is improved, thereby improving the response characteristics and sensitivity.

Moreover, combining with the hydrogen specificity of noble metal palladium (Pd), this application adopts in-situ growth technology and supercritical drying technology to realize in-situ growth of palladium ions into palladium nanoparticles under controllable conditions, and allow the palladium nanoparticles to be loaded on the “point-line” contact TiO₂ aerogel-hollow SnO₂ nanofiber heterojunction double-network composite framework aerogel.

The hydrogen sensor prepared herein is formed by encapsulating the composite aerogel mentioned above and the gold interdigital electrode. The gold interdigital electrode exhibits the multi-digit logarithm and can rapidly collect the resistance change signal of the composite aerogel. The series of hydrogen-sensing structure design, hydrogen-sensing target selection and preparation process has prospective significance for the development of excellent hydrogen-sensing materials, and has great prospects for the real-time monitoring of hydrogen in various fields.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a Fourier transform infrared spectrogram of polyvinylpyrrolidone (PVP)-tin dioxide (SnO₂) nanofiber and hollow SnO₂ nanofiber prepared in Example 1 according to this application;

FIGS. 2a-b are scanning electron microscope (SEM) images of the PVP-SnO₂ nanofiber prepared in Example 1 according to this application, where 2a: 2 μm (scale bar); and 2b: 200 nm (scale bar);

FIGS. 2c-d are SEM images of the hollow SnO₂ nanofiber prepared in Example 1 according to this application, where 2c: 2 μm (scale bar); and 2d: 200 nm (scale bar);

FIGS. 3a-b are SEM images of the PVP-SnO₂ nanofiber prepared in Example 2 according to this application, where 3a: 2 μm (scale bar); and 3b: 200 nm (scale bar);

FIGS. 3c-d are SEM images of the hollow SnO₂ nanofiber prepared in Example 2 according to this application, where 3(c): 2 μm (scale bar); and 3(d): 200 nm (scale bar);

FIG. 4 is an X-ray diffraction (XRD) pattern of the PVP-SnO₂ nanofiber and the hollow SnO₂ nanofiber prepared in Example 1 according to this application;

FIG. 5 shows a specific surface area of the PVP-SnO₂ nanofiber and the hollow SnO₂ nanofiber prepared in Example 1 according to this application, where abscissa indicates a relative pressure (dimensionless), and ordinate represents unit mass volume (unit: cubic centimeter per gram);

FIG. 6 shows a specific surface area of a palladium nanoparticle-loaded heterojunction double-network composite framework aerogel prepared in Example 3 according to this application, where abscissa indicates a relative pressure (dimensionless), and ordinate represents unit mass volume (unit: cubic centimeter per gram);

FIG. 7a is an SEM image of a heterojunction double-network composite framework aerogel loaded with 0.02 g of palladium nanoparticle prepared in Example 3 according to this application;

FIG. 7b is an SEM image of a heterojunction double-network composite framework aerogel loaded with 0.04 g of palladium nanoparticle prepared in Example 3 according to this application;

FIG. 7c is an SEM image of a heterojunction double-network composite framework aerogel loaded with 0.06 g of palladium nanoparticle prepared in Example 3 according to this application;

FIG. 8a is a curve showing sensitivity of a hydrogen sensor prepared in Example 4 to hydrogen with a concentration ranging from 100 to 1000 parts per million (ppm) and at 275° C., where the abscissa represents time (unit: second), the ordinate represents sensitivity, and pulse responses thereon (from left to right) respectively correspond to 100 ppm, 200 ppm, 300 ppm, 400 ppm, 500 ppm, 600 ppm, 700 ppm, 800 ppm, 900 ppm and 1000 ppm;

FIG. 8b is a curve showing sensitivity of the hydrogen sensor prepared in Example 4 to hydrogen with a concentration ranging from 100 ppm to 1000 ppm and at 300° C., where the abscissa represents time (unit: second), and the ordinate represents sensitivity;

FIG. 8c is a curve showing sensitivity of the hydrogen sensor prepared in Example 4 to change of hydrogen concentration from 100 ppm to 1000 ppm and at 325° C., where the abscissa represents time (unit: second), and the ordinate represents sensitivity;

FIG. 9 shows a response-recovery curve of a hydrogen sensor prepared in Example 5 at 300° C. when the hydrogen concentration is changed from 100 ppm to 1000 ppm;

FIG. 10 shows a sensitivity curve of the hydrogen sensor prepared in Example 5 at 300° C. when the hydrogen concentration is changed from 100 ppm to 1000 ppm;

FIG. 11 shows a concentration gradient curve of the hydrogen sensor prepared in Example 5 to the change of hydrogen concentration from 100 ppm to 1000 ppm at 300° C.;

FIG. 12 shows a sensitivity test curve of a pure titanium dioxide aerogel sensor to the change of hydrogen concentration from 100 ppm to 1000 ppm at 500° C.; and

FIG. 13 shows a response-recovery curve of the pure titanium dioxide aerogel sensor to the change of hydrogen concentration from 100 ppm to 1000 ppm at 500° C.

### DETAILED DESCRIPTION OF EMBODIMENTS

This application will be described in detail below with reference to the embodiments.

In order to clearly explain the objectives, technical solutions and advantages, the application is described in detail below with reference to the accompanying drawings and the following embodiments. The exemplary embodiments of this application and the description thereof are only intended to illustrate this application, and are not intended to limit the scope of this application.

As used herein, the room temperature refers to 25° C.

### Example 1

Provided herein was a method for preparing a palladium nanoparticle-loaded heterojunction double-network composite framework aerogel, which was performed as follows.

(Step 1) Preparation of Tin Dioxide (SnO₂) Nanofibers

0.4 g of tin dichloride monohydrate (SnCl₂·H₂O) was dissolved in 13-15 mL of a formamide-ethanol-acetone mixture (a volume ratio of formamide to ethanol to acetone was 2.5:2.5:1), and stirred for 30 min to form a transparent and clear SnO₂ precursor solution.

0.8 g of polyvinylpyrrolidone (PVP) powder was dissolved in the SnO₂ precursor solution, and heated under stirring at 50° C. for 5 h for complete dissolution of the PVP powder to form a transparent and viscous PVP-SnO₂ spinning solution.

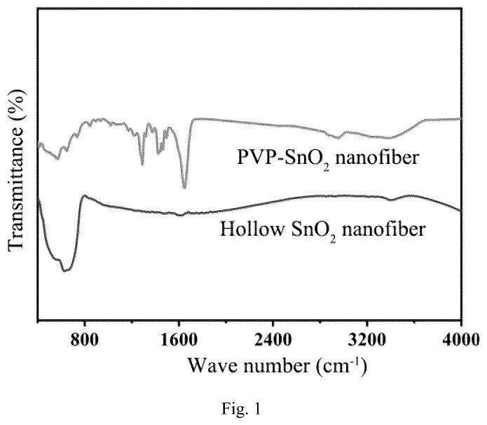

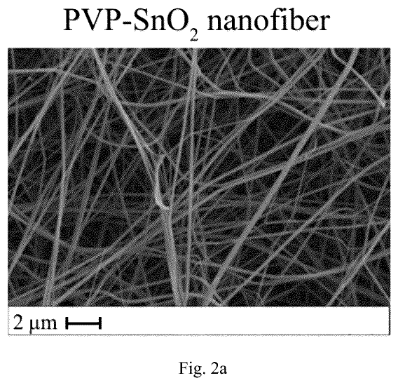

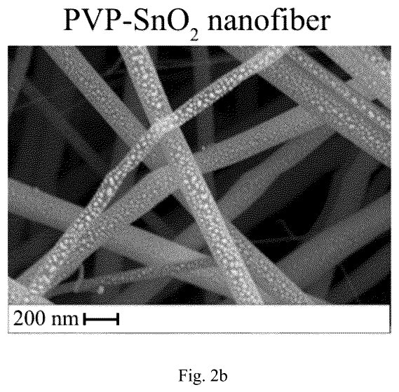

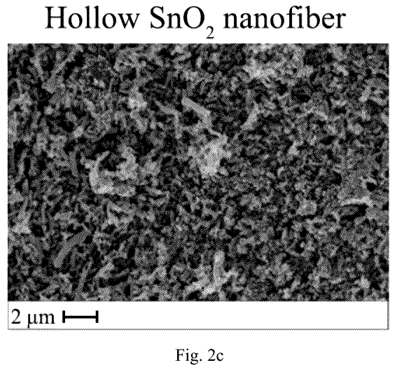

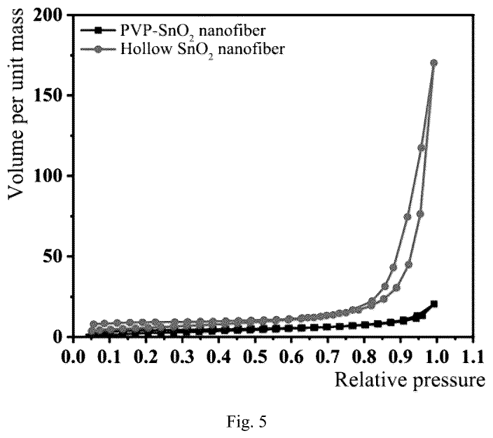

The PVP-SnO₂ spinning solution was loaded to a syringe (20 mL), and subjected to electrospinning at 25 kV and a speed of 6 μL/min to obtain a PVP-SnO₂ nanofiber mat, as shown in FIG. 1. The PVP-SnO₂ nanofiber mat had a fiber diameter of 20˜100 nm (as shown in FIGS. 2a-b), and a specific surface area of 13˜17 m²/g (as shown in FIG. 5).

The PVP-SnO₂ nanofiber mat was subjected to calcination in a temperature-programmed furnace at 500° C. for 2 h to obtain the hollow SnO₂ nanofiber. The hollow SnO₂ nanofiber has a single tetragonal structure, as shown in FIG. 1. The hollow SnO₂ nanofiber has a fiber diameter of 10-70 nm (shown in FIGS. 2c-d), and a specific surface area of 36˜42 m²/g (shown in FIG. 5).

(Step 2) Preparation of Tetrabutyl Titanate-Hollow SnO₂ Nanofiber Mixed Solution

The hollow SnO₂ nanofiber was fully ground, then added to a tetrabutyl titanate-absolute ethanol mixture, and stirred at room temperature to form a uniform “point-line” contact tetrabutyl titanate-hollow SnO₂ nanofiber mixed solution. A volume ratio of tetrabutyl titanate to absolute ethanol in the tetrabutyl titanate-absolute ethanol mixture was 1:23.

(Step 3) Preparation of Palladium Dichloride (PdCl₂) Precursor Solution

0.02˜0.06 g of PdCl₂ powder was dissolved in concentrated hydrochloric acid (concentration of 6 mol/L), and subjected to standing for 10 min to obtain a chloropalladium acid solution (orange-brown and transparent).

A formamide-ethanol-deionized water mixture was added to the chloropalladium acid solution, and stirred at room temperature for 2 h to obtain a uniform orange-brown transparent solution.

0.12˜0.36 g of PVP powder were added to the orange-brown transparent solution, and vigorously stirred at room temperature, and subjected to ultrasonic dispersion at a power of 100 W to obtain a transparent and clear orange-yellow PdCl₂ precursor solution with a pH ranging from 1.9 to 4.7.

(Step 4) Preparation of “Point-Line” Contact TiO₂ Aerogel-Hollow SnO₂ Nanofiber Heterojunction Double-Network Composite Framework Gel

Under vigorous stirring at room temperature, the PdCl₂ precursor solution was dropwise added to the tetrabutyl titanate-hollow SnO₂ nanofiber mixed solution at a rate of 1-2 drops/s. After 30 min, a crude gel was initially formed. The crude gel was subjected to aging for 2-3 days and solvent replacement by using anhydrous ethanol for 4-5 times (each time for 24 h) to form the “point-line” contact TiO₂ aerogel-hollow SnO₂ nanofiber heterojunction double-network composite framework gel with certain strength and uniformity.

(Step 5) Preparation of Palladium Nanoparticle-Loaded Heterojunction Double-Network Composite Framework Aerogel

The “point-line” contact TiO₂ aerogel-hollow SnO₂ nanofiber heterojunction double-network composite framework gel was put in a supercritical drying kettle, and completely immersed with absolute ethanol (liquid level of absolute ethanol was 3-4 centimeters higher than the gel). The supercritical drying kettle was adjusted to be at 45° C. and a pressure of 10˜14 MPa to allow carbon dioxide gas inside the supercritical drying kettle to reach a supercritical fluid state.

The pressure was regulated in the supercritical drying kettle, such that a three-dimensional (3D) network structure of the heterojunction double-network composite framework gel was maintained in carbon dioxide gas. The heterojunction double-network composite framework gel was subjected to degassing-pressure holding treatment five times (each pressure holding operation lasted for 1 h) to allow palladium ions to grow into palladium nanoparticles in situ under controllable conditions, such that the solvent inside the heterojunction double-network composite framework gel was replaced with air, thereby forming a palladium nanoparticle-loaded heterojunction double-network composite framework aerogel. The palladium nanoparticle had a particle size of 10˜20 nm. The heterojunction double-network composite framework gel had a pore size of 7˜30 nm, a specific surface area of 500-1000 m²/g, and a density of 0.1599˜0.2159 g/cm³.

(Step 6) Preparation of Hydrogen Sensor

A gold target is sputtered at a working distance of 25 mm and an electric current of 10 mA to generate gold ions. The gold ions are allowed to pass through a shadow mask to reach an aluminum oxide substrate and form a complete gold film on the aluminum oxide substrate, so as to form the gold interdigital electrode. The gold interdigital electrode has a size of 10 mm*10 mm. The number of pairs of the gold interdigital electrodes was 20. Moreover, the gold interdigital electrode has a line spacing of 50 μm, a line width of 80 m, and a length of 7.5 mm.

The palladium nanoparticle-loaded heterojunction double-network composite framework aerogel was ground to obtain a nano powder. The nano powder was mixed with deionized water to obtain a coating.

The gold interdigital electrodes were placed under a printed board. The coating was poured on the printed board. The hydrogen sensor is obtained through silk-screen printing and device aging.

### Example 2

Provided herein was a method for preparing a palladium nanoparticle-loaded heterojunction double-network composite framework aerogel, which was performed as follows.

(Step 1) Preparation of Tin Dioxide (SnO₂) Nanofibers

0.5 g of tin dichloride monohydrate (SnCl₂·H₂O) was dissolved in 13-15 mL of a formamide-ethanol-acetone mixture (a volume ratio of formamide to ethanol to acetone was 2.5:2.5:1), and stirred for 30 min to form a transparent and clear SnO₂ precursor solution.

1 g of polyvinylpyrrolidone (PVP) powder was dissolved in the SnO₂ precursor solution, and heated under stirring at 50° C. for 5 h for complete dissolution of the PVP powder to form a transparent and viscous PVP-SnO₂ spinning solution.

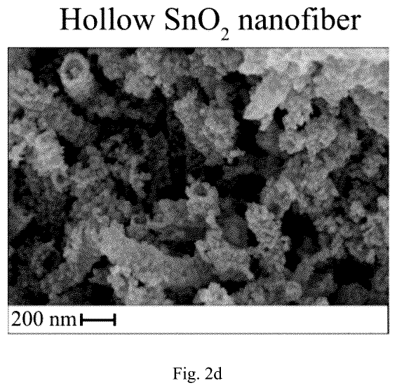

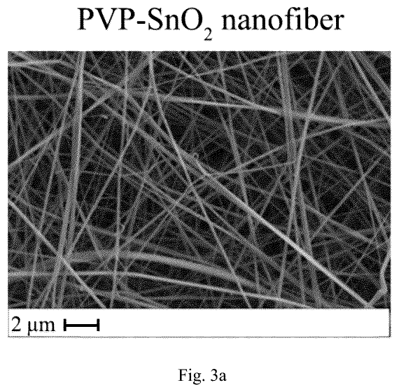

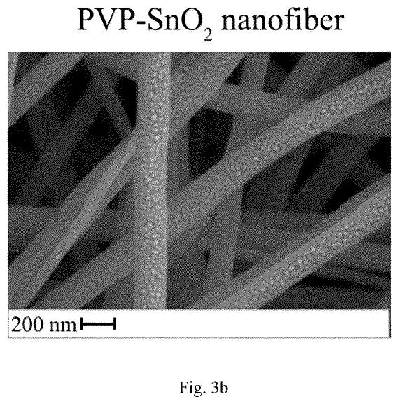

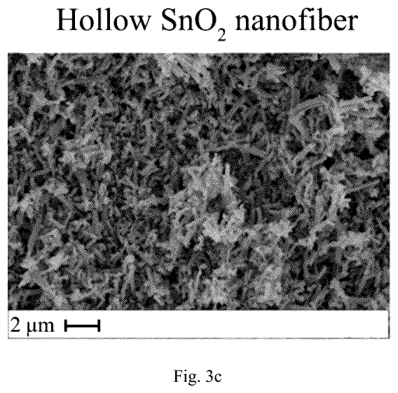

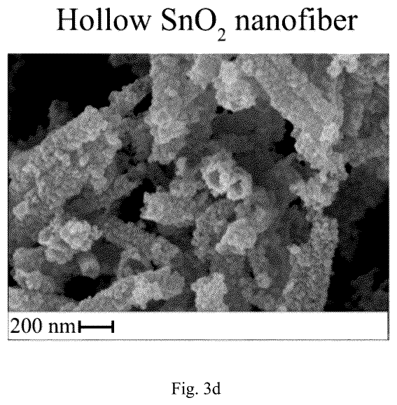

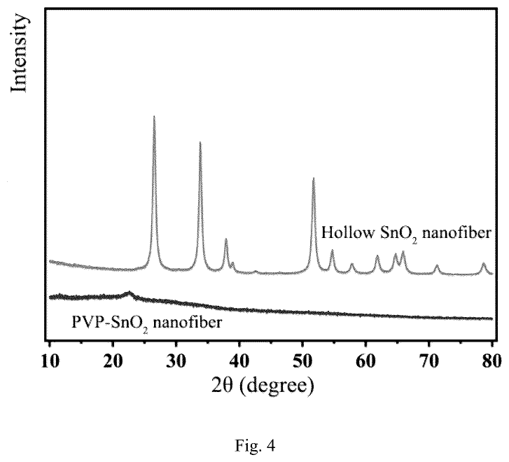

The PVP-SnO₂ spinning solution was added to a syringe (20 mL), and subjected to electrospinning at 25 kV and a speed of 6 μL/min to obtain a PVP-SnO₂ nanofiber mat. The PVP-SnO₂ nanofiber mat had a fiber diameter of 60˜180 nm (shown in FIGS. 3(a)-(b)), and a specific surface area of 14˜20 m²/g (as shown in FIG. 5).

The PVP-SnO₂ nanofiber mat was subjected to calcination in a temperature-programmed furnace at 500° C. for 2 h to obtain the hollow SnO₂ nanofiber. The hollow SnO₂ nanofiber has a single tetragonal structure. The hollow SnO₂ nanofiber had a fiber diameter of 50-120 nm (as shown in FIGS. 3c-d), and a specific surface area of 40-50 m²/g.

(Step 2) Preparation of Tetrabutyl Titanate-Hollow SnO₂ Nanofiber Mixed Solution

The hollow SnO₂ nanofiber was fully ground, then added to a tetrabutyl titanate-absolute ethanol mixture, and stirred at room temperature to form a uniform “point-line” contact tetrabutyl titanate-hollow SnO₂ nanofiber mixed solution. A volume ratio of tetrabutyl titanate to absolute ethanol in the tetrabutyl titanate-absolute ethanol mixture was 1:23.

(Step 3) Preparation of Palladium Dichloride (PdCl₂) Precursor Solution

0.02˜0.06 g of PdCl₂ powder was dissolved in concentrated hydrochloric acid (concentration of 6 mol/L), and subjected to standing for 10 min to obtain a chloropalladium acid solution (orange-brown and transparent).

A formamide-ethanol-deionized water mixture was added to the chloropalladium acid solution, and stirred at room temperature for 2 h to obtain a uniform orange-brown transparent solution.

0.12-0.36 g of PVP powder were added to the orange-brown transparent solution, and vigorously stirred at room temperature, and subjected to ultrasonic dispersion at a power of 100 W to obtain a transparent and clear orange-yellow PdCl₂ precursor solution with a pH ranging from 1.9 to 4.7.

(Step 4) Preparation of “Point-Line” Contact TiO₂ Aerogel-Hollow SnO₂ Nanofiber Heterojunction Double-Network Composite Framework Gel

Under vigorous stirring at room temperature, the PdCl₂ precursor solution was dropwise added to the TiO₂ aerogel-hollow SnO₂ nanofiber heterojunction double-network composite framework solution at a rate of 1-2 drops/s. After 30 min, a crude gel was initially formed. The crude gel was subjected to aging for 2-3 days and solvent replacement with anhydrous ethanol for 4-5 times (each for 24 h) to form the “point-line” contact TiO₂ aerogel-hollow SnO₂ nanofiber heterojunction double-network composite framework gel with high strength and uniformity.

(Step 5) Preparation of Palladium Nanoparticle-Loaded Heterojunction Double-Network Composite Framework Aerogel

The “point-line” contact TiO₂ aerogel-hollow SnO₂ nanofiber heterojunction double-network composite framework gel was put in a supercritical drying kettle, and completely immersed with absolute ethanol (liquid level of absolute ethanol was 3-4 centimeters higher than the gel). The supercritical drying kettle was adjusted to be at 45° C. and a pressure of 10˜14 MPa to allow carbon dioxide gas inside the supercritical drying kettle to reach a supercritical fluid state.

The pressure was regulated in the supercritical drying kettle, such that a three-dimensional (3D) network structure of the heterojunction double-network composite framework gel was maintained in carbon dioxide gas. The heterojunction double-network composite framework gel was subjected to degassing-pressure holding treatment five times (each pressure holding operation lasted for 1 h) to allow palladium ions to grow into palladium nanoparticles in situ under controllable conditions, such that the solvent inside the heterojunction double-network composite framework gel was replaced with air, thereby forming a palladium nanoparticle-loaded heterojunction double-network composite framework aerogel. The palladium nanoparticle had a particle size of 10˜20 nm. The heterojunction double-network composite framework gel had a pore size of 7˜30 nm, a specific surface area of 500-1000 m²/g, and a density of 0.1599˜0.2159 g/cm³.

(Step 6) Preparation of Hydrogen Sensor

A gold target was sputtered at a working distance of 25 mm and an electric current of 10 mA to generate gold ions. The gold ions were allowed to pass through a shadow mask to reach an aluminum oxide substrate and form a complete gold film on the aluminum oxide substrate, so as to form the gold interdigital electrode. The gold interdigital electrode had a size of 10 mm*10 mm. The number of pairs of the gold interdigital electrodes was 20. Moreover, the gold interdigital electrode had a line spacing of 50 μm, a line width of 80 μm, and a length of 7.5 mm.

The palladium nanoparticle-loaded heterojunction double-network composite framework aerogel was ground to obtain a nano powder. The nano powder was mixed with deionized water to obtain a coating.

The gold interdigital electrodes were placed under a printed board. The coating was poured on the printed board. The hydrogen sensor is obtained through silk-screen printing and device aging.

### Example 3

In this example, the hollow SnO₂ nanofiber obtained in Example 2 was adopted to prepare the heterojunction double-network composite framework aerogels loaded with 0.02 g, 0.04 g, and 0.06 g of palladium nanoparticles, respectively, according to the preparation method in Examples 1-2.

(Step 1) Preparation of Tin Dioxide (SnO₂) Nanofibers

0.5 g of tin dichloride monohydrate (SnCl₂·H₂O) was dissolved in 13-15 mL of a formamide-ethanol-acetone mixture (a volume ratio of formamide to ethanol to acetone was 2.5:2.5:1), and stirred for 30 min to form a transparent and clear SnO₂ precursor solution.

1 g of polyvinylpyrrolidone (PVP) powder was dissolved in the SnO₂ precursor solution, and heated under stirring at 50° C. for 5 h for complete dissolution of the PVP powder to form a transparent and viscous PVP-SnO₂ spinning solution.

The PVP-SnO₂ spinning solution was added to a syringe (20 mL), and subjected to electrospinning at 25 kV and a speed of 6 μL/min to obtain a PVP-SnO₂ nanofiber mat. The PVP-SnO₂ nanofiber mat had a fiber diameter of 60˜180 nm (as shown in FIGS. 3a-b), and a specific surface area of 14˜20 m²/g (as shown in FIG. 5).

The PVP-SnO₂ nanofiber mat was subjected to calcination in a temperature-programmed furnace at 500° C. for 2 h to obtain the hollow SnO₂ nanofiber. The hollow SnO₂ nanofiber has a single tetragonal structure. The hollow SnO₂ nanofiber has a fiber diameter of 50-120 nm, and a specific surface area of 40-50 m²/g.

(Step 2) Preparation Tetrabutyl Titanate-Hollow SnO₂ Nanofiber Mixed Solution

The hollow SnO₂ nanofiber was fully ground, then added to a tetrabutyl titanate-absolute ethanol mixture, and stirred at room temperature to form a uniform “point-line” contact tetrabutyl titanate-hollow SnO₂ nanofiber mixed solution. A volume ratio of tetrabutyl titanate to absolute ethanol in the tetrabutyl titanate-absolute ethanol mixture was 1:23.

(Step 3) Preparation of a Series of Palladium Dichloride (PdCl₂) Precursor Solutions

0.02 g of PdCl₂ powder was dissolved in 0.5 mL of concentrated hydrochloric acid (6 mol/L) and subjected to standing for 10 min to obtain a first chloropalladium acid solution (orange-brown transparent); 0.04 g of PdCl₂ powder was dissolved in 1.0 mL of concentrated hydrochloric acid (6 mol/L) and subjected to standing for 10 min to obtain a second chloropalladium acid solution; 0.06 g of PdCl₂ powder was dissolved in 1.5 mL of concentrated hydrochloric acid (6 mol/L) and subjected to standing for 10 min to obtain a third chloropalladium acid solution (containing 0.06 g of PdCl₂).

The first, second and third chloropalladium acid solutions were added with a formamide-ethanol-deionized water mixture, stirred at 25° C. for 2 h, respectively added with 0.12 g, 0.24 g and 0.36 g of PVP powder, vigorously stirred at room temperature, and subjected to ultrasonic dispersion at 100 W to obtain a first precursor solution (pH 1.9), a second precursor solution (pH 3.5) and a third precursor solution (pH 4.7).

(Step 4) Preparation of “Point-Line” Contact TiO₂ Aerogel-Hollow SnO₂ Nanofiber Heterojunction Double-Network Composite Framework Gels

Under vigorous stirring at room temperature, the first precursor solution (pH 1.9) was added to the “point-line” contact tetrabutyl titanate-hollow SnO₂ nanofiber mixed solution at a rate of 1-2 drops/s. After 30 min, a first crude gel was formed. Under vigorous stirring at room temperature, the second precursor solution (pH 3.5) was added to the “point-line” contact TiO₂ aerogel-hollow SnO₂ nanofiber heterojunction double-network composite framework solution at a rate of 1-2 drops/s. After 30 min, a second crude gel was formed. Under vigorous stirring at room temperature, the third precursor solution (pH 4.7) was added to the “point-line” contact TiO₂ aerogel-hollow SnO₂ nanofiber heterojunction double-network composite framework solution at a rate of 1-2 drops/s. After 30 min, a third crude gel was formed. The first, second and third crude gels were respectively subjected to aging for 2-3 days and solvent replacement by using anhydrous ethanol for 4-5 times (each time for 24 h) to respectively form a first “point-line” contact TiO₂ aerogel-hollow SnO₂ nanofiber heterojunction double-network composite framework gel, a second “point-line” contact TiO₂ aerogel-hollow SnO₂ nanofiber heterojunction double-network composite framework gel and a third “point-line” contact TiO₂ aerogel-hollow SnO₂ nanofiber heterojunction double-network composite framework gel.

(Step 5) Preparation of Heterojunction Double-Network Composite Framework Aerogels Respectively Loaded with 0.02 g, 0.04 g and 0.06 g of Palladium Nanoparticles

The first, second and third “point-line” contact TiO₂ aerogel-hollow SnO₂ nanofiber heterojunction double-network composite framework gels were respectively put in a supercritical drying kettle, and completely immersed with absolute ethanol (liquid level of absolute ethanol was 3-4 centimeters higher than the gel). The supercritical drying kettle was adjusted to be at 45° C. and a pressure of 10˜14 MPa to allow carbon dioxide gas inside of the supercritical drying kettle to reach a supercritical fluid state.

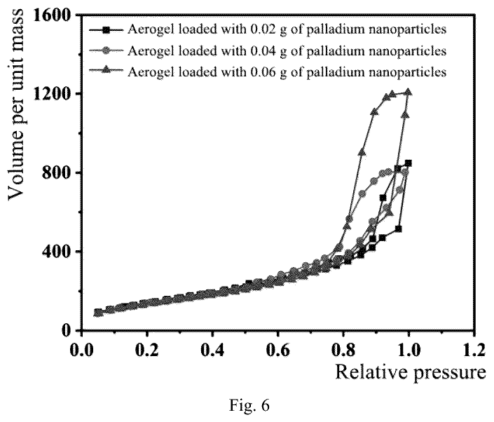

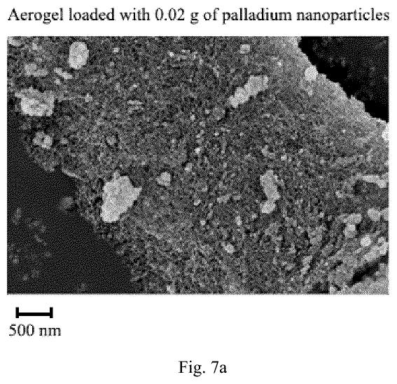

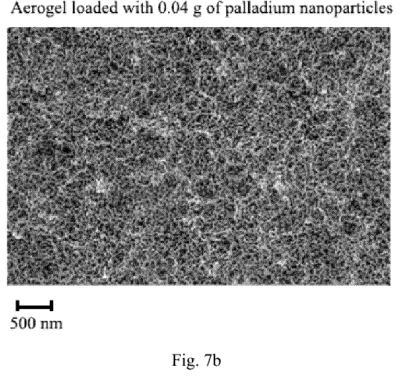

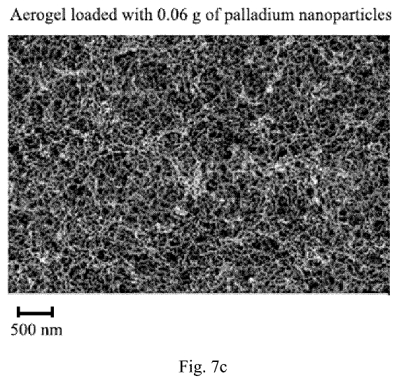

The pressure was regulated in the supercritical drying kettle, such that a three-dimensional (3D) network structure of each of the first, second and third heterojunction double-network composite framework gels was maintained in carbon dioxide gas. The first, second and third “point-line” contact TiO₂ aerogel-hollow SnO₂ nanofiber heterojunction double-network composite framework gels were respectively subjected to degassing-pressure holding treatment five times (each pressure holding operation lasted for 1 h) to allow palladium ions to grow into palladium nanoparticles in situ under controllable conditions, such that the solvents inside each of the first, second and third heterojunction double-network composite framework gels were replaced with air, thereby respectively forming a first heterojunction double-network composite framework aerogel loaded with 0.02 g of palladium nanoparticles, a second heterojunction double-network composite framework aerogel loaded with 0.04 g of palladium nanoparticles and a third heterojunction double-network composite framework aerogel loaded with 0.06 g of palladium nanoparticles, as shown in FIGS. 7(a)-(c). The palladium nanoparticle had a particle size of 10˜20 nm. The first heterojunction double-network composite framework aerogel loaded with 0.02 g of palladium nanoparticles, the second heterojunction double-network composite framework aerogel loaded with 0.04 g of palladium nanoparticles and the third heterojunction double-network composite framework aerogel loaded with 0.06 g of palladium nanoparticles each had a pore size of 7˜30 nm, a specific surface area of 500-1000 m²/g (as shown in FIG. 6), and a density of 0.1599˜0.2159 g/cm³.

(Step 6) Preparation of a Series of Hydrogen Sensors with Different Contents

A gold target is sputtered at a working distance of 25 mm and an electric current of 10 mA to generate gold ions. The gold ions are allowed to pass through a shadow mask to reach an aluminum oxide substrate and form a complete gold film on the aluminum oxide substrate, so as to form the gold interdigital electrode. The gold interdigital electrode has a size of 10 mm*10 mm. The number of pairs of the gold interdigital electrodes was 20. Moreover, the gold interdigital electrode has a line spacing of 50 μm, a line width of 80 μm, and a length of 7.5 mm.

The first heterojunction double-network composite framework aerogel loaded with 0.02 g of palladium nanoparticles, the second heterojunction double-network composite framework aerogel loaded with 0.04 g of palladium nanoparticles and the third heterojunction double-network composite framework aerogel loaded with 0.06 g of palladium nanoparticles were respectively ground to obtain a first nano powder, a second nano powder, and a third nano powder. The first, second and third nano powders were respectively fully mixed and ground by adding 1 mL of deionized water to obtain a first coating, a second coating and a third coating.

The gold interdigital electrodes were placed under a printed board. The first, second and third coatings were respectively poured onto the three printed board, and respectively subjected to silk-screen printing and aging to obtain a first hydrogen sensor made of the first heterojunction double-network composite framework aerogel loaded with 0.02 g of palladium nanoparticles, a second hydrogen sensor of the second heterojunction double-network composite framework aerogel loaded with 0.04 g of palladium nanoparticles and a third hydrogen sensor of the third heterojunction double-network composite framework aerogel loaded with 0.06 g of palladium nanoparticles

### Example 4

In this example, the test for optimal temperature of the hydrogen sensor made of the heterojunction double-network composite framework aerogel loaded with 0.06 g of palladium nanoparticles was performed as follows.

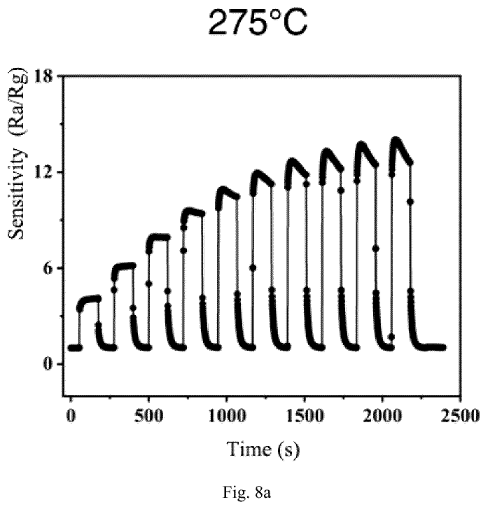

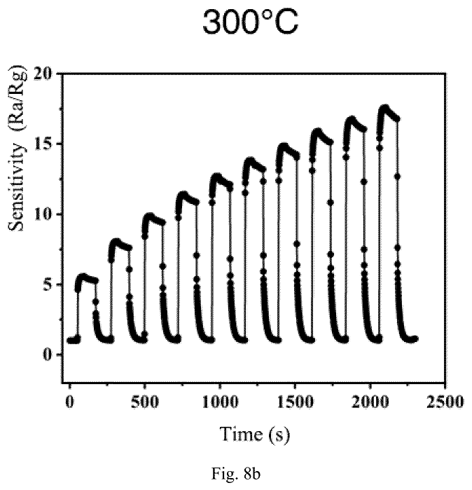

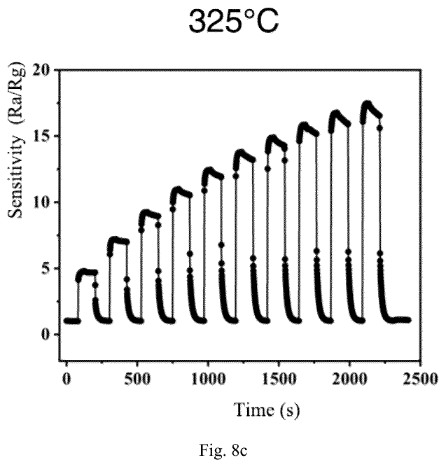

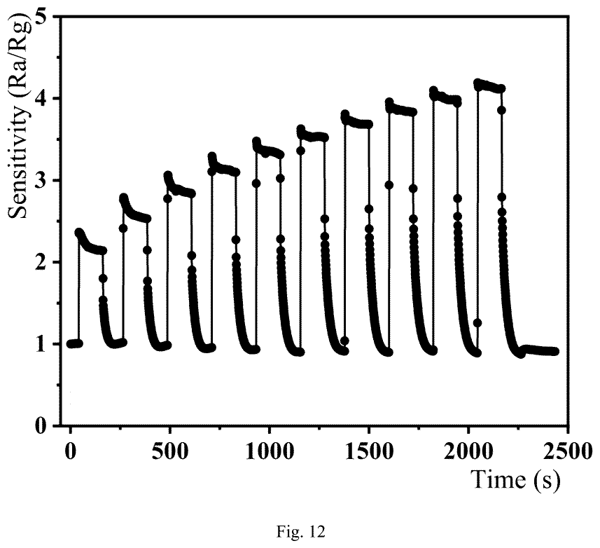

The above-mentioned hydrogen sensor was placed on a heating platform (set at 275-325° C.). The concentration range of the hydrogen was set to be within 100 ppm-1000 ppm. The ventilation time of hydrogen was 120 s, and the ventilation time of air was 100 s. The test for hydrogen sensitivity was performed, and the test results were demonstrated in FIGS. 8(a)-(c). FIG. 12 illustrated test results of the hydrogen concentration measured by the pure titanium dioxide aerogel sensor at 500° C. (300° C. was used in the embodiment of this application).

By comparing FIGS. 8a-c and FIG. 12, at 300° C., the hydrogen sensor provided herein had higher sensitivity to hydrogen. Therefore, 300° C. was taken as the optimal working temperature for the hydrogen sensor provided herein, at which the sensitivity was 6.1. Additionally, comparing with the pure titanium dioxide aerogel (optimal working temperature was 500° C., and sensitivity at 500° C. was 2.25), the hydrogen sensor provided herein significantly reduces the working temperature of the gas-sensitive material, and improves the optimal sensitivity.

### Example 5

In this example, the tests for response and recovery curve, sensitivity and concentration gradient of the hydrogen sensor made of the heterojunction double-network composite framework aerogel loaded with 0.06 g of palladium nanoparticles at 300° C. were performed as follows.

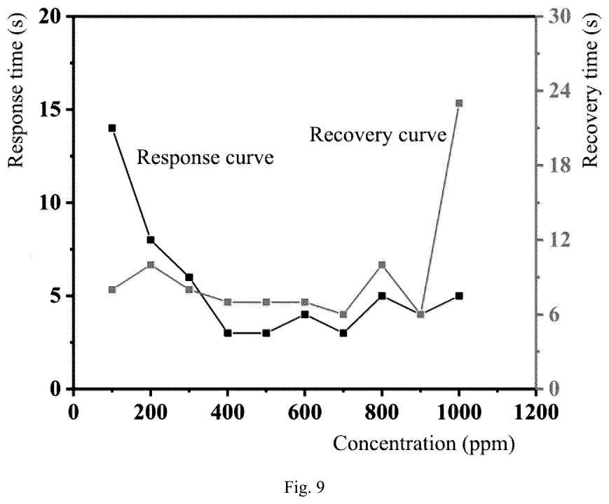

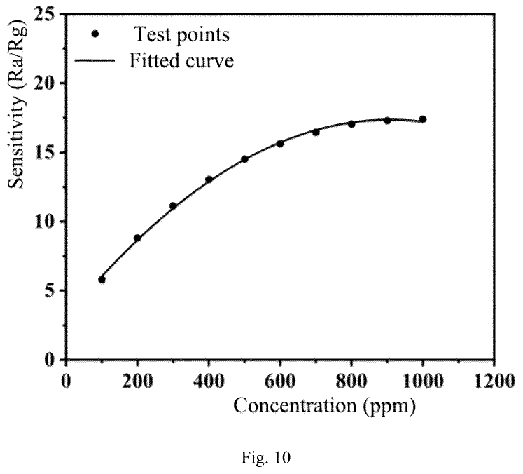

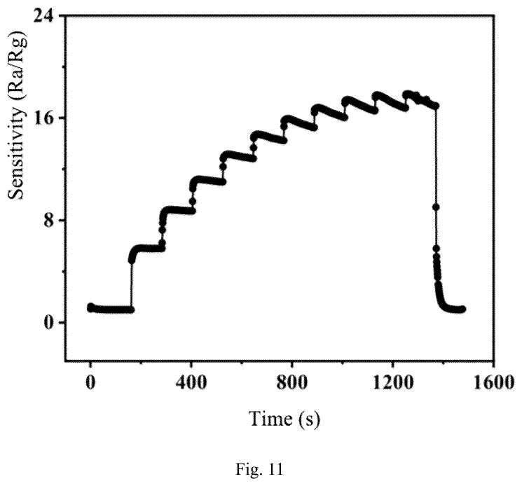

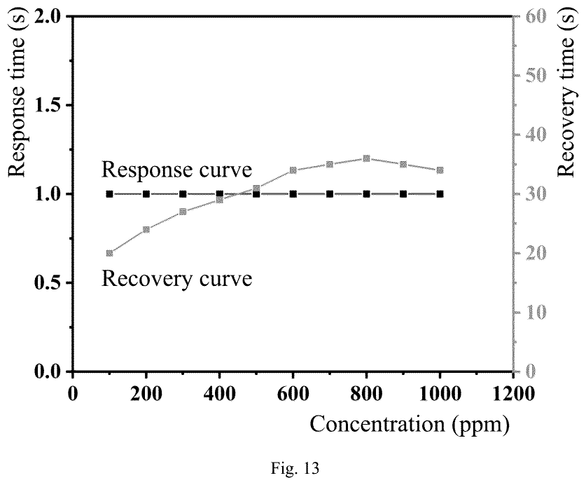

The above-mentioned hydrogen sensor was placed on a heating platform (set at 300° C.). The concentration range of the hydrogen was set to be within 100 ppm-1000 ppm. The ventilation time of hydrogen was 120 s, and the ventilation time of air was 100 s. FIGS. 9-11 respectively illustrated test results of gas sensitivity, response-recovery curve and sensitivity. Compared with FIGS. 12-13, it can be found that FIGS. 9-11 shows that the response time of the hydrogen sensor provided herein was about 2.5 s, which was similar to the response time (1 s) of pure titanium dioxide aerogel sensor. However, the recovery time of the hydrogen sensor provided herein was about 6 s, which was much shorter than the recovery time (35 s) of pure titanium dioxide aerogel sensor. In this case, the hydrogen sensor provided herein had significantly improved the optimal working temperature, sensitivity and recovery-response time. Based on the test results demonstrated above, this application had more excellent technical solution, structural design and gas-sensitivity performance.

Described above are intended to describe the objectives, technical solutions and beneficial effects of this application in detail. It should be understood that the above-mentioned embodiments are only illustrative of this application, and not intended to limit the scope of this application. Various modifications, equivalent replacements and improvements made without departing from the spirit and scope of this application shall fall within the scope of this application defined by the appended claims.

The embodiments described above are preferred embodiments of this application, which are not intended to limit the scope of the application. The preferred embodiments can be arbitrarily used in conjunction with or in combination with each other. The above embodiments with specific parameters therein are only intended for clearly expressing verification process of this application, and are not to limit the scope of this application. The scope of this application is still determined by the claims. Equivalent structural changes made in the specification and drawings of this application should be included in the protection scope of this application in the same way.

What is claimed is:

1. A method for preparing a palladium-loaded heterojunction composite framework aerogel, comprising:
(1) preparing a hollow tin dioxide (SnO₂) nanofiber;
(2) grinding the hollow SnO₂ nanofiber followed by addition of a tetrabutyl titanate-absolute ethanol mixture and stirring at room temperature to form a tetrabutyl titanate-hollow SnO₂ nanofiber mixed solution; wherein a volume ratio of tetrabutyl titanate to absolute ethanol in the tetrabutyl titanate-absolute ethanol mixture is 1:(20-25); and a mass ratio of the hollow SnO₂ nanofiber to the tetrabutyl titanate-absolute ethanol mixture is 1:(50-100);
(3) preparing a palladium dichloride (PdCl₂) precursor solution with a pH ranging from 1.9 to 4.7;
(4) dropwise adding the PdCl₂ precursor solution prepared in step (3) to the tetrabutyl titanate-hollow SnO₂ nanofiber mixed solution prepared in step (2) at a rate of 1-2 drops/s under stirring at room temperature until a crude gel is formed; and subjecting the crude gel to aging and multiple solvent replacements with anhydrous ethanol to form a heterojunction double-network composite framework gel; and
(5) placing the heterojunction double-network composite framework gel inside a supercritical drying kettle, followed by immersion in absolute ethanol, and adjusting a temperature and pressure in the supercritical drying kettle to allow carbon dioxide gas inside the supercritical drying kettle to reach a supercritical fluid state;
regulating the pressure in the supercritical drying kettle such that a three-dimensional (3D) network structure of the heterojunction double-network composite framework gel is maintained in carbon dioxide gas; subjecting the heterojunction double-network composite framework gel to degassing and pressure holding to allow palladium ions to grow into palladium nanoparticles in situ, so as to form a palladium nanoparticle-loaded heterojunction double-network composite framework aerogel.

2. The method of claim 1, wherein in step (1), the palladium-loaded heterojunction composite framework aerogel is prepared through the following steps:
dissolving tin dichloride monohydrate (SnCl₂·H₂O) in a formamide-ethanol-acetone mixture, followed by stirring to form a SnO₂ precursor solution; wherein a volume ratio of formamide to ethanol to acetone in the formamide-ethanol-acetone mixture is 2.5:2.5:1; and a weight-volume ratio of the SnCl₂·H₂O to the formamide-ethanol-acetone mixture is 1 (g) 32-38 (mL);
dissolving polyvinylpyrrolidone (PVP) powder in the SnO₂ precursor solution followed by heating at 45-55° C. under stirring for at least 4 h for complete dissolution of the PVP powder to form a PVP-SnO₂ spinning solution; wherein a weight ratio of SnCl₂·H₂O to the PVP powder is (2-3):1;
subjecting the PVP-SnO₂ spinning solution to electrospinning to obtain a PVP-SnO₂ nanofiber mat, wherein the PVP-SnO₂ nanofiber mat has a fiber diameter of 20˜100 nm, and a specific surface area of 13˜17 m²/g; and
subjecting the PVP-SnO₂ nanofiber mat to calcination in a temperature-programmed furnace at 480-515° C. to obtain the hollow SnO₂ nanofiber, wherein the hollow SnO₂ nanofiber has a single tetragonal structure.

3. The method of claim 1, wherein in step (3), the PdCl₂ precursor solution is prepared through steps of:
dissolving PdCl₂ powder in concentrated hydrochloric acid followed by standing to obtain a chloropalladium acid solution;
adding a formamide-ethanol-deionized water mixture into the chloropalladium acid solution, followed by stirring at room temperature for 2 h to obtain an orange-brown transparent solution; wherein a weight ratio of the PdCl₂ powder to the concentrated hydrochloric acid is 1:(1-5); and a volume ratio of formamide to ethanol to deionized water in the formamide-ethanol-deionized water mixture is 1:(13˜16):(2˜2.5); and
adding PVP powder into the orange-brown transparent solution followed by stirring at room temperature and ultrasonic dispersion to obtain the PdCl₂ precursor solution.

4. A method for preparing a hydrogen sensor, comprising:
preparing a gold interdigital electrode;
preparing a palladium-loaded heterojunction composite framework aerogel according to the method of claim 1; grinding the palladium-loaded heterojunction composite framework aerogel to obtain a nano powder; mixing the nano powder with deionized water to obtain a coating, wherein a weight ratio of the nano powder to the deionized water is 1:(10˜20); and
evenly applying the coating to the gold interdigital electrode to obtain the hydrogen sensor.

5. The method of claim 4, wherein the gold interdigital electrode is prepared by ion sputtering; wherein the ion sputtering is performed through steps of:
sputtering a gold target at a working distance of 25 mm and an electric current of 10 mA to generate gold ions; and allowing the gold ions to pass through a shadow mask to reach an aluminum oxide substrate and form a gold film on the aluminum oxide substrate, so as to form the gold interdigital electrode.
